(19) United States
(12) Patent Application Publication
(10) Pub. No.: US 2026/0027038 A1
(43) Pub. Date: Jan. 29, 2026

(54) Topical Skin Care Compositions

(71) Applicant: Allergan Sales, LLC, North Chicago, IL (US)

(72) Inventors: Tony T. Wang, Laguna Woods, CA (US); Soonjin Hong, Centennial, CO (US); Caroline Aguilar, San Marcos, CA (US); Prithwiraj Maitra, Ladera Ranch, CA (US)

(73) Assignee: Allergan Sales, LLC, North Chicago, IL (US)

(21) Appl. No.: 19/283,577

(22) Filed: Jul. 29, 2025

Related U.S. Application Data
(60) Provisional application No. 63/676,730, filed on Jul. 29, 2024.

(51) Int. Cl. A61K 8/9789; A61K 8/36; A61K 8/37; A61K 8/65; A61K 8/73; A61K 8/92; A61K 8/9794; A61Q 17/00; A61Q 19/00; A61Q 19/08

(52) U.S. Cl. CPC A61K 8/9789; A61K 8/361; A61K 8/37; A61K 8/65; A61K 8/735; A61K 8/922; A61K 8/9794; A61Q 17/00; A61Q 19/00; A61Q 19/007; A61Q 19/08

(57) ABSTRACT

The present disclosure relates to topical skin care compositions that increase the hydration of aging skin, improve the barrier function of aging skin, provide antioxidant protection to aging skin and enhance dermal-epidermal junction health in aging skin.

### RELATED APPLICATION

This application claims the benefit of U.S. Application Ser. No. 63/676,730, filed Jul. 29, 2024, the contents of which are incorporated herein by reference in its entirety.

### FILED OF THE INVENTION

The present invention relates to topical compositions and methods for skincare treatment employing the topical compositions disclosed herein. More specifically, the topical compositions and methods for skincare disclosed herein are cosmetic formulations that may be applied to various areas of human skin, including but not limited to face and neck areas to treat signs of aging such as wrinkles, fine lines and sagging skin.

### BACKGROUND

Normal healthy skin has a smooth epidermal layer that acts as a strong barrier to water and environmental damage. Natural firming and hydrating elements such as collagen (which provides skin firmness), elastin (which supplies skin elasticity and rebound) and glycosaminoglycans or GAGs (which keep the skin hydrated) are abundant in normal healthy skin. Aging can have a negative effect on the health, function and appearance of skin.

Aging caused by the natural aging process is sometimes referred to as intrinsic aging. Intrinsic aging is a continuous process that normally begins in the mid-twenties. Aging slows the natural production of collagen, elastin, and GAGs within the skin, which results in unwanted changes in skin such as dryness, itchiness, redness, shadows or dark areas, sagging, thinning, or more noticeable fine lines and wrinkles. Aging also slows the natural production of adenosine triphosphate (ATP), a nucleotide known to drive and participate in intracellular processes including repairs. Dermal levels of ATP decline with age and create an energy deficiency, thereby affecting dermal function and skin appearance.

Aging caused by environmental factors such as sun exposure, chemicals, microorganisms, food or environmental pollutants can accelerate the aging process. Aging due to environmental factors is sometimes referred to as extrinsic aging, which can trigger a variety of enzymatic and non-enzymatic modifications in skin. One such non-enzymatic process is the reaction of proteins with reducing sugars (glycation or Maillard reaction). Glycation alters the structure and functional properties of proteins, including collagen, elastin, and GAGs, and adversely affects cellular metabolism. The accumulation of glycation products is observed in skin during aging.

Aging generally causes a decrease in a subject's extracellular matrix (“ECM”) and its major component hyaluronic acid (“HA”). HA stabilizes the intracellular structures by forming a viscoelastic network in which collagen and elastin fibers are embedded. HA provides a cushion effect to the skin structures including the epidermis. The loss of HA and consequently the viscoelastic buffering system contributes to tearing, resulting in skin disruptions and skin dehydration leading to visible wrinkles on stratum corneum.

Destruction and/or loss of dermal collagen fiber also contributes to skin wrinkling and increased appearance of muscular attachments.

The biological signs of aging are apparent in both the epidermis layer and dermis layer of the skin and include a decrease of epidermal thickness (that correlates with the reduced keratinocyte proliferation), a reduction and disorganization of major key components, such as HA, collagens and other elastic fibers (proteoglycans and glycosaminoglycans), an increase in matrix metalloproteinases production and a flattening of the dermal-epidermal junction (“DEJ”).

With age, skin homeostasis is also disrupted, metabolic activities are slowed, and endogenous production and accumulation of reactive oxygen species (ROS) leading to oxidative cellular stress and secretion of inflammatory mediators may occur.

The prior art is replete with various topical compositions for treating the signs of aging skin. Some such compositions are described in U.S. Pat. Nos. 11,224,566 and 11,696,888 which are incorporated herein by reference. Examples of commercially available topical products that have been or are marketed for treating the effects of skin aging include SKINMEDICA's HA⁵® Rejuvenating Hydrator, ALASTIN's HA (Hyaluronic acid) IMMERSE SERUM™, ALGENIST's GENIUS LIQUID COLLAGEN®, ELEMIS' PRO-COLLAGEN MARINE CREAM, PCA SKIN's COLLAGEN HYDRATOR CREAM, SKINCEUTICALS' Hyaluronic Acid Intensifier, and SKINCEUTICALS' Hydrating B5 Gel.

Although numerous topical anti-aging compositions are described in the art, there remains a need to develop an improved topical anti-aging, skin care composition.

### SUMMARY OF THE INVENTION

The present invention is a topical composition that may be applied to various skin areas of a human subject to repair damage caused by intrinsic and/or extrinsic aging factors. The topical composition comprises: (i) one or more collagens; (ii) one or more barrier support agents; (iii) one or more bio-hydration support agents; (iv) one or more dermal-epidermal junction (“DEJ”) agents; (v) one or more antioxidants; and (vi) HA, HA derivatives or combinations of HA and HA derivatives.

The topical composition may be a solution, suspension, dispersion, emulsion, gel, cream, lotion, ointment or serum and further comprise one or more conventional topical/cosmetic carriers and excipients such as one or more solvents, skin conditioning agents (sometimes referred to as humectants, moisturizers or emollients), preservatives, thickeners/viscosity enhancing agents, pH adjusting agents, buffering agents, chelating agents, surfactants, solubilizing agents or emulsifying agents and combinations of the foregoing.

In certain embodiments, the topical composition comprises: (i) about 0.0001 wt % to about 2.5 wt % of one or more collagens; (ii) about 0.0001 wt % to about 5 wt % of one or more barrier support agents; (iii) about 0.0001 wt % to about 5 wt % of one or more bio-hydration support agents; (iv) about 0.0001 wt % to about 5 wt % of one or more DEJ agents; (v) about 0.001 wt % to about 2.5 wt % of one or more antioxidants; (vi) about 0.001 wt % to about 2.5 wt % of HA, HA derivatives or combinations of HA and HA derivatives and (vii) one or more conventional topical/cosmetic carriers and excipients such as one or more solvents, skin conditioning agents (sometimes referred to as humectants, moisturizers or emollients), preservatives, thickeners/viscosity enhancing agents, pH adjusting agents, buffering agents, chelating agents, surfactants, solubilizing agents or emulsifying agents and combinations of the foregoing. In certain aspects of this embodiment, the topical composition will further comprise at least about 60 wt % or more of water.

The topical composition may be applied to various skin areas of a human subject, particularly areas of the face, neck and décolletage area at least once or more times a day, preferably two, three or four times a day.

In certain embodiments the topical compositions may also be applied to various skin areas before, during or after an ancillary therapy, including but not limited to laser treatment, chemical peels, intense pulsed light, dermabrasion or cryotherapy.

The topical compositions are particularly useful as anti-aging treatments and will reduce or improve the appearance of horizontal lines, wrinkles, crepey-like skin, hyperpigmentation, redness, and sagging triggered by aging, dehydration, and other environmental factors.

### BRIEF DESCRIPTION OF THE FIGURES

FIG. 1 shows the results of the in vitro HA and proteoglycans tests described in Example 4.

FIG. 2A shows the results of the in vitro barrier function, cell-cell adhesion tests described in Example 4.

FIG. 2B shows the results of the in vitro barrier function, cornified envelope tests described in Example 4.

FIG. 2C shows the results of the in vitro barrier function, lipid synthesis tests described in Example 4.

FIG. 3 shows the results of the in vitro antioxidant tests described in Example 4.

FIG. 4 shows the results of the in vitro DEJ tests described in Example 4.

### DETAILED DESCRIPTION OF THE INVENTION

Before the present invention is further described, it is to be understood that this invention is not limited to the particular embodiments described. It is also to be understood that the terminology used herein is for the purpose of describing particular embodiments only, and is not intended to be limiting.

It should be noted that as used herein, the singular forms “a,” “an,” and “the” include plural referents unless the context clearly dictates otherwise.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

The present invention is a topical composition that may be applied to various skin areas of a human subject to repair damage caused by intrinsic and/or extrinsic aging factors. In particular, the topical compositions in accordance with the present invention unexpectedly exhibits improved hydration, improved cell-cell adhesion, improved barrier function improved antioxidant properties and enhanced DEJ properties compared to commercially available anti-aging products. In contrast, a significant challenge in developing topical compositions for treating naturally and/or environmentally aged skin is their ability to target specific areas of the epidermal and dermal layers of skin. The present inventors surprisingly discovered the combination of one or more collagens, barrier support agents, bio-hydration support agents, DEJ agents, antioxidants, and HA and/or HA derivatives in the topical compositions described herein target specific areas of the epidermal and dermal layers of the skin as demonstrated in gene test panels described more particularly herein.

In certain embodiments, the topical composition of the present invention can be used to: decrease and/or prevent the characteristics of wrinkles, small wrinkles and/or fine lines of the skin; improve and/or decrease the microrelief of the skin; smooth the skin; plump the skin; improve the density of the skin; maintain and/or restore skin elasticity; maintain and/or restore skin barrier integrity; maintain and/or restore skin firmness; maintain and/or restore the cohesion of skin compartments, in particular the cohesion of the derm within the skin; and combinations of the foregoing.

The collagens that may be employed in the present invention to aid in replenishing bio-humectants on the skin surface and help to promote a rapid or quick improvement in skin hydration and particularly hydration of the strateum corneum. As used herein, collagen refers to the main protein of connective tissue, which is found throughout the bodies of mammals and is of at least 12 types. The collagens can be either of a synthetic or natural origin. Examples of collagens that may be used in the present invention can be found in U.S. Pat. Nos. 4,973,473 and 6,146,650 and U.S. Patent Application Publication Nos. 2014/0148417 and 2022/0401525, all of which are incorporated herein by reference. In certain embodiments, the collagens are non-naturally occurring collagens such as those described in U.S. Pat. Nos. 11,028,148; 11,041,015; 11,166,126; 11,180,541; and 11,214,609, all of which are incorporated herein by reference. The non-naturally occurring collagens may be produced by a host cell. The non-naturally occurring collagens may be a truncated collagen.

The truncation is an internal truncation, a truncation at the N-terminal portion of the collagen, or a truncation at the C-terminal portion of the collagen. The collagen is truncated by a truncation of between 50 amino acids and 1000 amino acids, between, 50 amino acids and 950 amino acids, between 50 amino acids and 900 amino acids, between 50 amino acids and 850 amino acids, between 50 amino acids and 800 amino acids, between 50 amino acids and 850 amino acids, between 50 amino acids and 800 amino acids, between 50 amino acids and 750 amino acids, between 50 amino acids and 700 amino acids, between 50 amino acids and 650 amino acids, between 50 amino acids and 600 amino acids, between 50 amino acids and 650 amino acids, between 50 amino acids and 500 amino acids, between 50 amino acids and 450 amino acids, between 50 amino acids and 400 amino acids, between 50 amino acids and 350 amino acids, between 50 amino acids and 300 amino acids, between 50 amino acids and 250 amino acids, between 50 amino acids and 200 amino acids, between 50 amino acids and 150 amino acids, or between 50 amino acids and 100 amino acids. In another embodiment, the collagen or elastin is truncated by 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900, 950, or 1000 amino acids. An example of a preferred collagen is a synthetic human polypeptide-121 commercially available from Gelto, Inc. of Emeryville, California, USA. In a certain embodiment, the collagen is a synthetic collagen, wherein the synthetic collagen is collagen 21, preferably sh-polypeptide-121 available from Gelto, Inc. under the tradename HUMACOLL21®).

The one or more collagens are present in the topical compositions of the present invention in an amount of about 0.0001 wt % to about 2.5 wt % based on the total weight of the composition, preferably about 0.0005 wt % to about 2 wt % based on the total weight of the composition and more preferably about 0.001 wt % to about 1.5 wt % based on the total weight of the composition.

The barrier support agents that may be employed in the present invention are compounds that aid or support the barrier function of the skin. Examples of barrier support agents include saturated and unsaturated fatty acids and esters and salts thereof. Examples of barrier support agents include but are not limited to C₁₂ to C₂₀ fatty acids such as linoleic acid, linolenic acid, oleic acid, stearic acid, and palmitic acid, esters thereof, salts thereof and combinations of the foregoing. In certain embodiments the present invention includes at least two unsaturated fatty acids, preferably selected from linoleic acid, linolenic acid and oleic acid, esters thereof, salts thereof or combinations therefore. In certain embodiments the present invention comprises linoleic acid, linoleic salt, linoleic ester or combinations thereof and linolenic acid, linolenic salt, linolenic ester or combinations thereof wherein the linoleic acid, linoleic salt, linoleic ester or combination thereof is present in an amount that is at least 10 times, 20 times, 30 times, 40 times, 50 times, 60 times, 70, times, 80 times, 90 times, 100 times 110 times, 120 times, 130 times, 140 times greater than the amount of linolenic acid, linolenic salt, linolenic ester or combination thereof.

The one or more barrier support agents are present in the topical compositions of the present invention in an amount of about 0.0001 wt % to about 5 wt % based on the total weight of the composition, preferably about 0.00025 wt % to about 3 wt % based on the total weight of the composition and more preferably about 0.0005 wt % to about 2 wt % based on the total weight of the composition.

The bio-hydration support agents that may be employed in the present invention are compounds that aid or support the skin's natural bio-hydration. The compounds may aid or support the skin's natural bio-hydration by upregulating endogenous HA and proteoglycans and specifically collagen 15 and collagen 18. Examples of bio-hydration support agents include saw palmetto extract, black pepper leaf extract and vegetable and animal fats and oils such as castor oil, safflower oil, cotton seed oil, corn oil, olive oil, cod liver oil, almond oil, avocado oil, palm oil, sesame oil, and soybean oil as well as extracts of the oils and plants. In a certain embodiment, the bio-hydration support agent includes extracts and derivatives of the avocado fruit, also known as Persea grattisma such as those described in U.S. Pat. Nos. 3,846,556; 4,032,628; RE29,871; 4,297,374; 4,324,802, and 11,026,441. In certain embodiments the bio-hydration support agent comprises butyl avocadate which is an ester of butyl alcohol and the fatty acids derived from Persea grattisma (avocado) oil.

The one or more bio-hydration support agents are present in the topical compositions of the present invention in an amount of about 0.0001 wt % to about 5 wt % based on the total weight of the composition, preferably about 0.0025 wt % to about 3 wt % based on the total weight of the composition and more preferably about 0.0005 wt % to about 2 wt % based on the total weight of the composition.

The DEJ agents that may be employed in the present invention are compounds that promote the proliferation of the DEJ, improve epidermal and DEJ health and supports the various collagens that are present in the skin such as collagen 4, collagen 7 and collagen 17. In certain embodiments the DEJ agent is a compound, preferably a botanical extract, that has a composition similar to the main components of the skin dermis and/or DEJ such collagen, elastin and laminins. The DEJ agent may increase the ECM synthesis and/or inhibit ECM enzymes such as collagenases and elastases that catalyze the degradation or breakdown of collagen and elastin fibers. The DEJ agent may also reduce carbamylation that in turn prevents or deters the deterioration of collagen and other EMC and DEJ proteins and thereby improves the organization of collagen.

Examples of DEJ agents that may be employed in the present invention include, but are not limited, to Chlorella vulgaris extract as described in International Patent Application No. WO 2007/078056 and commercially available under the tradename CHLORELLAGEN, and mushroom extracts such as Lentinus edodes extract, commercially available under the tradename ACTIFICOL and Passiflora edulis seed oil examples of which are described in U.S. Pat. Nos. 11,596,662; 11,026,880 and 10,688,142 and commercially available under the tradename PASSIOLINE.

The one or more DEJ agents are present in the topical compositions of the present invention in an amount of about 0.0001 wt % to about 5 wt % based on the total weight of the composition, preferably about 0.0025 wt % to about 3 wt % based on the total weight of the composition and more preferably about 0.0005 wt % to about 2 wt % based on the total weight of the composition.

The antioxidants that may be employed in the present invention are compounds that retard oxidation or degradation. The antioxidant may retard the oxidation or degradation of one or more ingredients in the topical composition and/or may retard the oxidation or degradation of the components of the skin cells in the target area or application site. Unless specifically stated, the antioxidant may be a commonly known antioxidant such as those described in the United State Pharmacopcia or Handbook of Pharmaceutical Excipients, a botanical extract or a combination thereof.

Examples of antioxidants that may be employed in the present invention include, but are not limited to, niacinamide, peptides, such as di-, tri-, tetra and hexa-peptides, acetyl cysteine, ascorbic acid polypeptide, ascorbyl dipalmitate, ascorbyl methylsilanol pectinate, ascorbyl palmitate, ascorbyl stearate, butylated hydroxyanisole (BHA), butylated hydroxytoluene (BHT), t-butyl hydroquinone, cysteine, cysteine HCl, diamylhydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, Helianthus annus (sunflower) seed oil, hydroquinone, isooctyl thioglycolate, kojic acid, Laminaria digitata extract, magnesium ascorbate, magnesium ascorbyl phosphate, methylsilanol ascorbate, natural botanical anti-oxidants such as Camellia sinnensis green tea extract, Nelumbo nucifera leaf, flower or germ extract, Vitris vinifera (grape) callous culture extract, nordihydroguaiarctic acid, octyl gallate, phenylthioglycolic acid, potassium ascorbyl tocopheryl phosphate, potassium sulfite, propyl gallate, quinones, rosmarinic acid, Rosa mochata seed oil, Rosmarinus officinalis (rosemary) leaf extract, sodium ascorbyl phosphate, sodium ascorbate, sodium bisulfite, sodium erythorbate, sodium metabisulfite, sodium sulfite, superoxide dismutase, sodium thioglycolate, sorbityl furfural, thiodiglycol, thiodiglycolamide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, Tremella fuciformis (mushroom) extract, tris (nonylphenyl) phosphite, and Zea mays (corn) oil. In certain embodiments, the topical compositions may comprise one or more antioxidants selected from the group consisting of, Vitis vinifera flower cell extract, Nelumbo nucifera germ extract (also known as lotus seed extract), tocopherol acetate, acetyl tetrapeptide-2, lutein (also known as xanthophyll), propyl gallate, hydroxyacetaphenone, sodium bisulfite, diethylhexyl syringylidenemalonate, BHA, BHT, Citrullus lanatus (Watermelon) fruit extract, Chamomilla recutita (matricaria) flower extract or combinations thereof.

The one or more antioxidants are present in the topical compositions of the present invention in an amount of about 0.001 wt % to about 2.5 wt % based on the total weight of the composition, preferably about 0.0025 wt % to about 2 wt % based on the total weight of the composition and more preferably about 0.005 wt % to about 1.5 wt % based on the total weight of the composition.

As used herein the term “hyaluronic acid derivatives” or “HA derivatives” include but are not limited to hyaluronic salts, such as sodium hyaluronate, cross-linked hyaluronic acid and hydrolyzed hyaluronic acid. Examples of the HA and HA derivatives that may be used in the present invention are described in U.S. Pat. Nos. 11,696,888 and 11,224,566 and Narurkar et al., “A New Approach in Topical Hyaluronic Acid: Going Beyond Instant Benefits to Restore Epidermal HA Homeostasis,” Journal of Drugs in Dermatology, Vol. 15, Issue 1, Suppl. 2, pp. S23-S38 (January 2016), all of which are incorporated herein by reference.

The HA, HA derivatives or combinations thereof are present in the topical compositions of the present invention in an amount of about 0.001 wt % to about 2.5 wt % based on the total weight of the composition, preferably about 0.0025 wt % to about 2 wt % based on the total weight of the composition and more preferably about 0.005 wt % to about 1.5 wt % based on the total weight of the composition.

In certain embodiments, the topical composition includes 0.001 wt % to 1.5 wt % of one or more collagens; 0.0005 wt % to 2 wt % of one or more barrier support agents including linoleic acid, linolenic acid, or combinations thereof; 0.0005 wt % to 2 wt % of one or more bio-hydration support agents including a botanical 5α reductase inhibitor comprising butyl avocadate, saw palmetto extract, black pepper extract or combinations thereof; 0.0005 wt % to 2 wt % of a dermal-epidermal junction support agent including Passiflora edulis seed oil; 0.005 wt % to 1.5 wt % of one or more antioxidants including Nelumbo nucifera germ extract, Vitis vinifera (grape) flower extract or combinations thereof; and 0.005 wt % to 1.5 wt % of hyaluronic acid, hyaluronic acid derivatives, or combinations thereof including sodium hyaluronate, hydrolyzed hyaluronic acid, sodium hyaluronate crosspolymer or combinations thereof.

The topical compositions of the present invention may further comprise one or more conventional topical/cosmetic carriers and excipients such as one or more solvents, skin conditioning agents (sometimes referred to as humectants, moisturizers or emollients), preservatives, thickeners/viscosity enhancing agents, pH adjusting agents, buffering agents, chelating agents, surfactants, solubilizing agents or emulsifying agents and combinations of the foregoing. The CTFA International Cosmetic Ingredient Dictionary and Handbook (2008), 12th Edition, describes a wide variety of non-limiting cosmetic ingredients that may be used in embodiments of the present invention.

Examples of solvents that may be used in the topical compositions of the present invention include water and/or organic based solvents such as C₁-C₁₀ mono-alcohols (e.g., methanol, ethanol, isopropanol, benzyl alcohol, phenoxyethanol etc.) C₂-C₁₂ polyalcohols (e.g., ethylene glycol, propylene glycol, hexylene glycol, glycerin, etc.) or combinations thereof. In certain embodiments, the topical compositions of the present invention comprise about 30 wt % to about 97 wt %, preferably about 35 wt % to about 96 wt % and more preferably about 40 wt % to about 95 wt % of one or more solvents based on the total weight of the topical composition.

In certain embodiments the topical composition of the aspects of this embodiment, the topical composition will further comprise at least about 60 wt % or more of water, at least 65 wt % or more of water, at least 70 wt % or more of water, at least 75 wt % or more of water, at least 80 wt % or more of water, at least 85 wt % or more of water, or at least 90% or more of water.

In certain embodiments, the topical composition of the present invention may comprise one or more skin conditioning agents. As used herein skin conditioning agents include compounds that hydrate or improve the texture and feel of skin and include humectants, moisturizers and emollients. In certain embodiments, the topical composition will comprise about 0.0005 wt % to about 20 wt %, preferably about 0.0007 wt % to about 15 wt % and more preferably about 0.001 wt % to about 10 wt % based on the total weight of the topical composition of one or more skin conditioning agents. Examples of skin conditioning agents include but are not limited to, lactic acid and other hydroxy acids and their salts, glycerin, propylene glycol, butylene glycol, sodium pyrrolidine carboxylic acid (“PCA”), Carbowax 200, Carbowax 400, and Carbowax 800, PPG-15 stearyl ether, lanolin alcohol, lanolin, lanolin derivatives, cholesterol, petrolatum, isostearyl neopentanoate, octyl stearate, mineral oil, isocetyl stearate, Ceraphyl 424 (myristyl myristate), octyl dodecanol, dimethicone (Dow Corning 200-100 cps), phenyl trimethicone (Dow Corning 556), Dow Coning 1401 (cyclomethicone and dimethiconol), and cyclomethicone (Dow Corning 344), and Miglyol 840 (manufactured by Huls; propylene glycol dicaprylate/dicaprate) guanidine, urea, glycolic acid, glycolate salts (e.g. ammonium and quaternary alkyl ammonium), salicylic acid, lactic acid, lactate salts (e.g., ammonium and quaternary alkyl ammonium), aloe vera in any of its variety of forms (e.g., aloe vera gel), polyhydroxy alcohols such as sorbitol, mannitol, xylitol, erythritol, glycerol, hexanetriol, butanetriol, propylene glycol, butylene glycol, hexylene glycol and the like, polyethylene glycols, sugars (e.g., melibiose), starches, sugar and starch derivatives (e.g., alkoxylated glucose, fructose, glucosamine), hyaluronic acid, lactamide monoethanolamine, acetamide monoethanolamine, panthenol. Additional examples of skin conditioning agents can be found in U.S. Pat. Nos. 5,804,203 and 8,741,357, which are incorporated herein by reference. In certain embodiments, the topical compositions may comprise one or more skin conditioning agents selected from the group consisting of glycerin, coconut alkanes, Butyrospermum parkii (shea) butter, ethylhexyl olivate, polyisobutene, squalene, cetearyl alcohol, arginine, trehalose, coco-caprylate/caprate, ethylhexylglycerin, sucrose, caprylic/capric triglyceride, sodium hyaluronate, hyaluronic acid, sorbitan isostearate, cyclopentasiloxane, PEG-10 dimethicone, dimethicone/vinyl dimethicone crosspolymer, hydrated silica, ceramide, silica, Dunaliella salina extract, caprylyl glycol, 1-2-hexanediol, aloe barbadensis leaf extract, butylene glycol, panthenol, Lens esculenta (lentil) fruit extract, sodium PCA, Laminaria digitata extract, Artemisia vulgaris extract, isopentyldiol, betaine, Hamamelis virginiana (witch hazel) water, Avena sativa (oat) bran extract or combinations thereof.

Examples of preservatives that may be included in the topical compositions of the present invention include, but are not limited to, potassium sorbate, acids, alcohols, glycols, parabens, quaternary-nitrogen containing compounds, isothiazolinones, aldehyde-releasing compounds and halogenated compounds. Illustrative alcohols include phenoxyethanol, isopropyl alcohol, and benzyl alcohol; illustrative glycols include propylene, butylene and pentylene glycols; illustrative parabens include (also known as parahydroxybenzoic acids) methyl, propyl and butyl parabens; illustrative quaternary nitrogen containing compounds include benzalkonium chloride, Quartenium 15; illustrative isothiazoles include methylisothiazoline, methychlorolisothiazoline; illustrative aldehyde releasing agents include DMDM hydantion, imiadolidinyl urea and diazolidinyl urea; illustrative antioxidants include butylated hydroxytoluene, tocopherol and illustrative halogenated compounds include triclosan and chlorohexidene digluconate. In some embodiments, the compositions employ a combination of phenoxyethanol, chlorphenesin, and caprylyl glycol as preservatives.

Examples of viscosity enhancing agents that may be used in the topical compositions of the present invention include but are not limited to carboxylic acid polymers such as carbomers, which are homopolymers of acrylic acid crosslinked with allyl ethers of sucrose or pentaerytritol, available as under the tradename CARBOPOL®. In addition, other suitable carboxylic acid polymeric agents include ULTREZ® 10 and copolymers of C₁₀-C₃₀ alkyl acrylates with one or more monomers of acrylic acid, methacrylic acid, or one of their short chain (i.e., C₁-C₄) esters, wherein the crosslinking agent is an allyl ether of sucrose or pentaerytritol. These copolymers are known as acrylates/C₁₀-C₃₀ alkyl acrylate crosspolymers and are commercially available as CARBOPOL® 1342, CARBOPOL® 1382, PEMULEN TR-1, and PEMULEN® TR-2.

Additional viscosity enhancing agents that may also be used in the topical compositions of the present invention include crosslinked polyacrylate polymers, including both cationic and nonionic polymers, with the cationic being generally preferred. Examples of useful crosslinked nonionic polyacrylate polymers and crosslinked cationic polyacrylate polymers are those described in U.S. Pat. Nos. 5,100,660, 4,849,484, 4,835,206, 4,628,078, 4,599,379 and EP228868.

Further examples of viscosity enhancing agents that may also be used in the topical compositions of the present invention include a wide variety of polysaccharides. “Polysaccharides” refer to gelling agents that contain a backbone of repeating sugar (i.e., carbohydrate) units. Non-limiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, microcrystalline cellulose, sodium cellulose sulfate, and mixtures thereof. Also useful herein are the alkyl-substituted celluloses. Other useful polysaccharides include scleroglucans comprising a linear chain of (1-3) linked glucose units with a (1-6) linked glucose every three units, a commercially available example of which is CLEARGEL™ CS11 from Michel Mercier Products Inc.

Still further examples of viscosity enhancing agents that may also be used in the topical compositions of the present invention include thickening and/or gelling agents which are primarily derived from natural sources. Non-limiting examples of these naturally derived thickening and/or gelling agents include acacia, agar, algin, alginic acid, ammonium alginate, amylopectin, calcium alginate, calcium carrageenan, carnitine, carrageenan, dextrin, gelatin, gellan gum, guar gum, guar hydroxypropyltrimonium chloride, hectorite, hyaluronic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures thereof.

Additional examples of viscosity enhancing agents include silicone containing compounds, e.g., silicone oils, silicone elastomers and/or polyorganosiloxanes. Examples of the foregoing can be found in U.S. Pat. No. 10,286,030, which is incorporated herein by reference. In certain embodiments of the present invention the topical composition will comprise a silicone elastomer and/or polyorganosiloxanes, such as a dimethicone crosspolymer commercially available under the tradename DOWSIL™ EL-8040 ID Silicone Organic Blend, which will provide multiple properties including but not limited to increasing the viscosity and feel of the composition and provide skin conditioning benefits.

Examples of pH modifying agents that may be used in the topical compositions of the present invention include but are not limited to compounds that are added to raise or low the pH of the final topical composition as well as compounds that are added to buffer the final topical composition. Compounds that may be added to raise or lower the pH of the topical composition include but are not limited to phosphoric acid and/or phosphate salts, citric acid and/or citrate salts, hydroxide salts (i.e., calcium hydroxide, sodium hydroxide, potassium hydroxide) and amines, such as triethanolamine. Examples of compounds that buffer the topical compositions include, acetic acid and salts, citric acid and salts, boric acid and salts, and phosphoric acid and salts such as mono- and di-potassium phosphate, citric acid/sodium citrate, and dibasic sodium phosphate/citric acid.

Examples of chelating agents that may be used in the topical compositions of the present invention include but are not limited to citric acid and salts thereof, disodium EDTA, tetrasodium EDTA, and phytic acid.

Examples of surfactants, solubilizing agents or emulsifying agents such as decyl glucoside, polysorbates (i.e., polysorbate 20, 60 or 80) and combinations thereof.

Additional specific examples of the carriers, auxiliaries and/or excipients of the foregoing classes can be found in U.S. Pat. Nos. 9,408,881; 10,286,030; and 10,493,011 and U.S. Patent Application Publication Nos. 2011/0158922; 2012/0076842; and 2015/0202139, all of which are incorporated herein by reference. The skilled artisan is aware that some of the carries, auxiliaries and/or excipients may be included in more than one of the foregoing classifications. Stated another way, some of the carriers, auxiliaries and/or excipients may impart one or more properties to the topical composition.

In certain embodiments, the topical composition of the present invention may further comprise one or more anti-inflammatory agents. In certain embodiments, the topical composition will comprise about 0.0005 wt % to about 5.00 wt %, preferably about 0.0007 wt % to about 2.5 wt % and more preferably about 0.001 wt % to about 1.00 wt % based on the total weight of the topical composition of one or more anti-inflammatory agents. Examples of anti-inflammatory agents properties include but are not limited to dipotassium glycyrrhizate, bisabolol, allantoin, aloe extracts, panthenol, Chamomilla recutita (matricaria) flower extract and materials derived from the following: Phellodendron amurense corte extract (PCE), Tanacetum parthenium commercially available under the tradename FEVERFEW, Zingiber officinate (ginger) root extract, ginko (Ginkgo biloba), Centella asiatica extract, commercially available under the tradename MADECASSOSIDE, Cotinus (Cotinus coggygria), butterbur extract (Petasites hybridus), goji berry (Lycium barbarum), milk thistle extract (Silybum marianum), honeysuckle (Lonicera japonica), basalm of peru (Myroxylon pereirae), sage (Salvia officinalis), cranberry extract (Vaccinium oxycoccos), amaranth oil (Amaranthus cruentus), pomegranate (Punica granatum), yerbe mate (Ilex paraguariensis leaf extract), white lily flower extract (Lilium candidum), olive leaf extract (Olea europaea), phloretin (apple extract), oat flour (Avena sativa), hops (Humulus lupulus) extract, commercially available under the tradename LIFENOL, bugrane p (Ononis spinosa), licochalcone (licorice: Glycyrrhiza inflate extract ingredient), and ginger extract, commercially available under the tradename SYMRELIEF, and combinations thereof. Additional examples may be found in U.S. Patent Application Publication Nos. 2020/0069562 and 2017/0128357, which are incorporated herein by reference. In certain embodiments, the topical compositions may comprise one or more anti-inflammatory agents selected from the group consisting of dipotassium glycyrrhizate, bisabolol, allantoin, aloe leaf extracts, panthenol, Chamomilla recutita (matricaria) flower extract, Avena sativa bran extract, or combinations thereof.

In certain embodiments, the topical composition of the present invention may further comprise one or more exfoliants. In certain embodiments, the topical composition will comprise about 0.0005 wt % to about 5.00 wt %, preferably about 0.0007 wt % to about 2.5 wt % and more preferably about 0.001 wt % to about 1.00 wt % based on the total weight of the topical composition of one or more exfoliants. Examples of exfoliants include but are not limited to retinoids, alpha-hydroxy acids such as lactic acid, glycolic acid, malic acid, tartaric acid, citric acid, or any combination of any of the foregoing, and beta-hydroxy acids such as salicylic acid, polyhydroxy acids such as lactobionic acid and gluconic acid. Examples of the exfoliants can also be found in U.S. Pat. Nos. 5,756,107; 9,161,958 and 9,782,334, all of which are incorporated herein by reference.

Topical compositions of the present invention may be a lotion, emulsion, cream, gel, ointment, foam, liquid, paste or other topically administrable form. In certain embodiments, a topical composition is a serum, gel, or liquid. Embodiments of the topical compositions may exhibit a viscosity of less than 100,000 cps, preferably less than 75,000 cps and more preferably less than 50,000 cps at 25° C. when tested using a conventional viscosity apparatus, such as a Brookfield RVT viscometer with spindle 2 and 20 rpms. In certain embodiments, the topical composition will exhibit a viscosity at 25° C. between about 3,000 cps and 75,000 cps, preferably about 5,000 cps and about 60,000 cps and more preferably about 7,000 cps and about 50,000 cps. In certain embodiments, the topical composition will exhibit a low viscosity at 25° C. between about 1 cps and 200 cps, preferably about 1 cps and about 100 cps and more preferably about 1 cps and about 50 cps.

Topical compositions of the present invention may be prepared by any method commonly known in the industry and may include blending, mixing, emulsifying, heating, cooling steps or any combination thereof. Examples of manufacturing methods can be found in U.S. Pat. No. 10,493,011 and U.S. Patent Application Publication No. 2011/0158922, both of which are incorporated herein by reference.

Topical compositions of the present invention may be packaged and dispensed in any suitable container. Containers can include a bottle, a tube (metal, plastic or laminate), a pressurized container, or pouches. The containers may include amounts of the topical composition for single or multiple applications. Containers may include indicia on its surface that instruct the subject on its application and use. Instructions for use may also be printed separately and packaged with the container comprising the topical composition.

Containers comprising multiple applications of the topical composition can dispense a pre-determined amount of the topical composition via a metered or calibrated spray, pump, or squeeze mechanism. In other embodiments, the container can be squeezed (e.g., metal, laminate, or plastic tube) to dispense a desired amount of the composition.

Lower viscosity topical compositions, i.e., topical compositions with viscosity of less than 200 cps, may be applied to the skin using a pad or wipe. A pad or wipe may be made of cotton, woven or non-woven polymeric material such as polyester, polypropylene, nylon, rayon or combinations thereof. The low viscosity topical composition can be applied to a pad or wipe and one or more pads or wipes may be packaged in a suitable container such as a jar, plastic bottle, or flexible packaging such as a single use foil package. The lower viscosity compositions may also be employed in conjunction with a procedure conducted in a dermatologist or skin-care professional office (i.e., an in-office procedure), such as a chemical peel, laser/light therapy, micro needling and/or microdermabrasion.

In certain embodiments, the subject in need of or desiring treatment will apply the desired amount of the topical composition to a clean and preferably dry skin surface with at least once a day, preferably twice or thrice a day. In certain embodiments, the subject will apply about 0.1 to about 2.0 grams, preferably 0.20 grams to about 1.5 grams and more preferably about 0.3 grams to about 1.6 grams. The amount will be adjusted depending upon the surface area to be treated. In some embodiments, the topical composition in accordance with the present invention may be applied to the target area once, twice or thrice daily for at least 1 day, 2 days, 3 days, 4 days, 5 days, 6 days, 1 week, 2 weeks, 3 weeks, 1 month, 2 months, 3 months, 4 months, 5 months, 6 months, or more.

The topical compositions as described herein may be used during a procedure conducted in a dermatologist or skin-care professional office (i.e., an in-office procedure). The in-office procedure include but are not limited to a chemical peel, laser/light therapy, micro needling and/or microdermabrasion. After the in-office procedure, the patient/subject will use the same or different topical composition as described herein to further treat the disorders while at home or out of the office. For example, a first topical composition as described herein may be used immediately before, during, or immediately after a microdermabrasion procedure. In this context, immediately before or immediately after means within 30, 25, 20, 15, 10, 5 minutes of the in-office procedure. Once the in-office procedure is completed, the patient/subject will apply one or more of the topical compositions described herein to the treatment area, once, twice, thrice or more times a day while at home or out of the office for a period of time such as 1, 2, 3, 4, 5, 6, 7, 8, 9, 10, 11, 12 or more weeks. The described in-office and out of office treatment process may be repeated as necessary or desired.

Although any methods and materials similar or equivalent to those described herein can also be used in the practice of the present invention, the preferred methods and materials are now described. All publications mentioned herein are incorporated herein by reference to disclose and describe the methods and/or materials in connection with which the publications are cited.

The following examples are provided by way of illustration only and are by no means intended to be limiting.

### Example 1

Topical compositions in accordance with the present invention may be prepared having the following:

| | WEIGHT PERCENT | | |
|---|---|---|---|
| INGREDIENT | Composition A | Composition B | Composition C |
| Collagens | 0.0001-2.5 | 0.0005-2.0 | 0.001-1.5 |
| Barrier Support Agents | 0.0001-5 | 0.00025-3 | 0.0005-2 |
| Bio-Hydration Support | 0.0001-5 | 0.00025-3 | 0.0005-2 |
| Agents | | | |
| DEJ Agents | 0.0001-5 | 0.00025-3 | 0.0005-2 |
| Antioxidants | 0.001-2.5 | 0.0025-2.0 | 0.005-1.5 |
| HA-HA Derivatives | 0.001-2.5 | 0.0025-2 | 0.005-1.5 |

The topical compositions summarized in the above table may be a solution, suspension, dispersion, emulsion, gel, cream, lotion, ointment or serum and further comprise conventional topical/cosmetic carriers and excipients such as one or more solvents, skin conditioning agents, preservatives, thickeners/viscosity enhancing agents, pH adjusting agents, buffering agents, chelating agents, surfactants, solubilizing agents or emulsifying agents and combinations of the foregoing.

In certain embodiments of the topical compositions summarized in the above table, the collagen comprises a synthetic collagen, such as collagen 21 (also known as sh-polypeptide-121 available from Geltor, Inc. under the tradename HUMACOLL21®)

In certain embodiments of the topical compositions summarized in the above table, the barrier support agents comprises linolenic acid, linoleic acid or a combination thereof.

In certain embodiments of the topical compositions summarized in the above table, the bio-hydration support agents comprises natural 5-α reductase inhibitors such as butyl avocadate, saw palmetto extract, black pepper extract and combinations thereof.

In certain embodiments of the topical compositions summarized in the above table, the DEJ agents comprises one or more botanical extracts such as Chlorella vulgaris extract, mushroom extracts such as Lentinus edodes extract, Passiflora edulis seed oil or a combination thereof.

In certain embodiments of the topical compositions summarized in the above table, the antioxidant is selected from the group consisting of Nelumbo nucifer germ extract, Vitis vinifera flower cell extract, tocopherol, tocopherol salts and esters such as tocopherol acetate, acetyl tetrapeptide-2, lutein (also known as xanthophyll), propyl gallate, hydroxyacetaphenone, sodium bisulfite, diethylhexyl syringylidenemalonate, BHA, BHT, Citrullus lanatus (watermelon) fruit extract, Chamomilla recutita (matricaria) flower extract or combinations thereof.

In certain embodiments of the topical compositions summarized in the above table, the composition comprises a mixture of HA and HA derivatives such as salts of HA, e.g. sodium hyaluronate, cross-linked HA and hydrolyzed HA or combinations thereof.

In certain embodiments of the topical compositions summarized in the above table, the compositions may further comprise one or more conventional topical/cosmetic carriers and excipients selected from the group consisting of water, glycerin, caprylic/capric triglyceride, Shorea stenoptera seed butter, Butyrospermum parkii (shea) butter, C₉-C₁₂ alkanes, propanediol, sodium acrylates copolymer, glyceryl glucoside, 1,2-hexanediol, lecithin, trehalose, urea, oleic acid, palmitic acid, coco-caprylate/caprate, pentylene glycol, caprylhydroxamic acid, serine, stearic acid, polyglutamic acid, algin, capryl glycol, glyceryl polyacrylate, pullulan, one or more polysorbates, potassium phosphate, tocopherol, sodium benzoate, aloe barbadensis leaf extract, one or more vitamin B and/or provitamin B (e.g. panthenol), allantoin, phenoxyethanol, chlorphenesin, caprylyl glycol, PPG-2 isoceteth 20 acetate or combinations thereof.

### Example 2

Topical compositions in accordance with the present invention and particularly topical serum are prepared having the following composition:

| Ingredient | Weight Percent |
|---|---|
| Water | 50-90 |
| Glycerin | 0.5-15 |
| Linoleic Acid | 0.1-5 |
| Passiflora Edulis Seed Oil | 0.05-5 |
| Butyl avocadate | 0.01-2 |
| Nelumbo Nucifera Germ Extract | 0.001-2 |
| Vitis Vinifera (Grape) Flower Cell | 0.001-2 |
| Extract | |
| Linolenic Acid | 0.001-1 |
| Sodium Hyaluronate | 0.001-2 |
| Sodium Hyaluronate Crosspolymer | 0.001-1 |
| Hydrolyzed Hyaluronic Acid | 0.001-1 |
| Collagen 21 | 0.0001-0.5 |
| Oleic Acid | 0.01-2 |
| Palmitic Acid | 0.01-2 |
| Stearic Acid | 0.001-2 |
| Polyglutamic Acid | 0.001-2 |

The topical compositions described in the above table may further comprise caprylic/capric triglyceride, Shorea stenopetera seed butter, C₉-C₁₂ alkanes Propanediol, sodium acrylates coplomyer, glyceryl glucoside, 1,2-hexanediol, lecithin, trehalose, urea, coco-caprylate/caprate, pentylene glycol, caprylhydroxamic acid, serine, algin, caprylyl glycol, disodium phosphate, glyceryl polyacrylate, pullulan, polysorbate 20, potassium phosphate tocopherol and sodium benzoate.

The above topical composition may be filled into a suitable multi-use container that can dispense a pre-determined amount of the topical composition via a metered or calibrated spray, pump, or squeeze mechanism for application to a subject's treatment area one, two three or more times a day for as long as the treatment is desired or beneficial.

### Example 3

Topical compositions in accordance with the present invention and particularly topical liquids that are useful before during or after a procedure conducted in a dermatologist or skin-care professional office such as during a microdermabrasion procedure are prepared having the following composition:

| Ingredient | Weight Percent |
|---|---|
| Water | 75-98 |
| Glycerin | 0.1-15 |
| Aloe Barbadensis Leaf Extract | 0.001-1 |
| Panthenol | 0.01-3 |
| Allantoin | 0.001-1 |
| Linoleic Acid | 0.0001-1 |
| Passiflora Edulis Seed Oil | 0.0001-1 |
| Butyl Avocadate | 0.0001-1 |
| Nelumbo Nucifera Germ Extract | 0.0001-1 |
| Vitis Vinifera (Grape) Flower Cell | 0.001-2 |
| Extract | |
| Linolenic Acid | 0.0001-1 |
| Sodium Hyaluronate | 0.0001-1 |
| Sodium Hyaluronate Crosspolymer | 0.0001-1 |
| Hydrolyzed Hyaluronic Acid | 0.001-1 |
| Collagen 21 | 0.0001-0.5 |
| Oleic Acid | 0.0001-1 |
| Palmitic Acid | 0.0001-1 |
| Stearic Acid | 0.0001-1 |
| Polyglutamic Acid | 0.001-2 |

The topical compositions described in the above table may further comprise butylene glycol, disodium EDTA, pentylene glycol, trehalose, urea, serine, algin, caprylyl glycol, disodium phosphate, glyceryl polyacrylate, pullulan, polysorbate 20, potassium phosphate, caprylhydroxamic acid, 1,2-hexanediol, phenoxyethanol, chlorphenesin, lecithin, benzyl alcohol, benzoic acid, dihyroacetic acid, glyceryl glucoside, tocopherol, sodium benzoate and PPG-2 isoceteth-20 acetate.

### Example 4

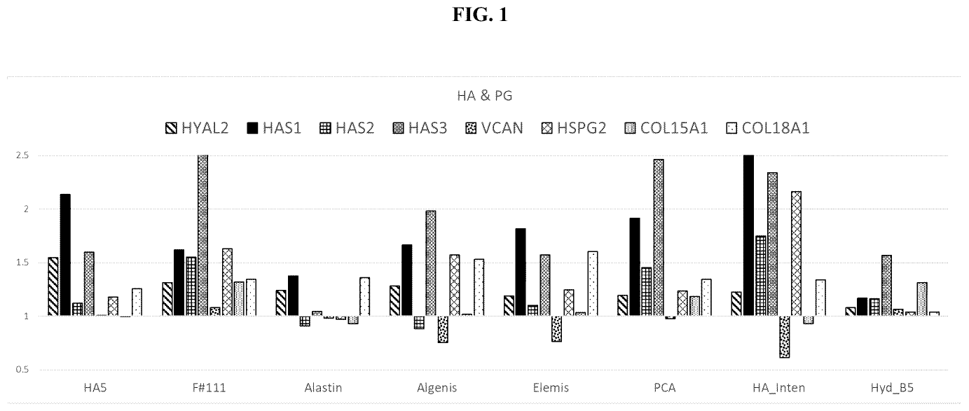

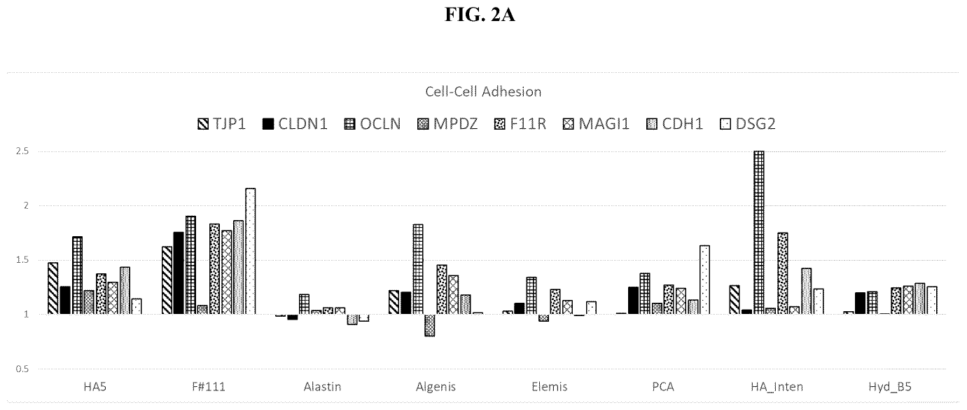

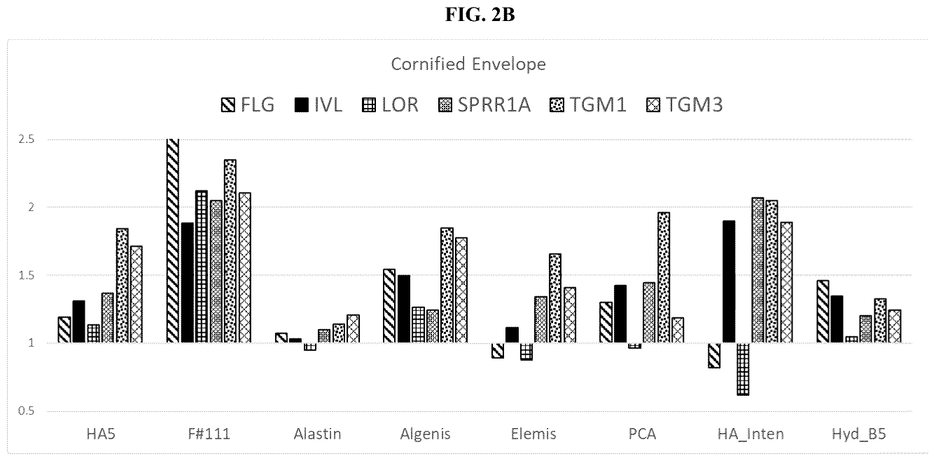

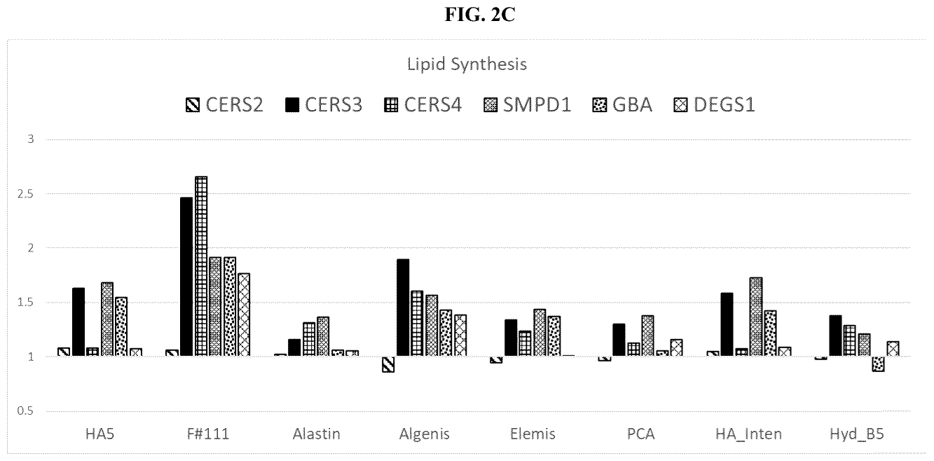

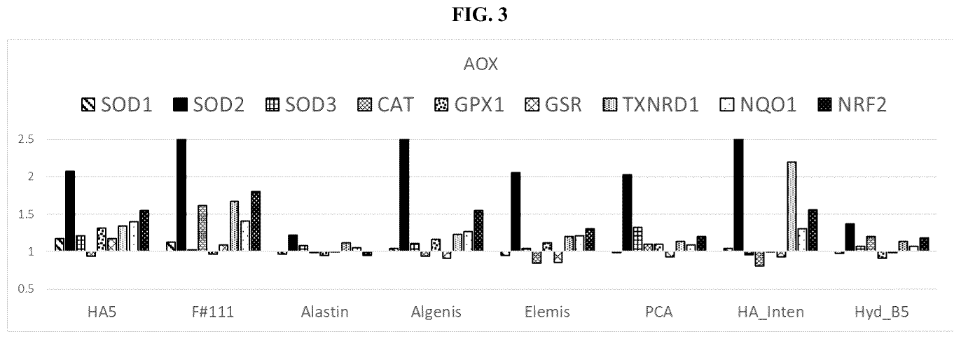

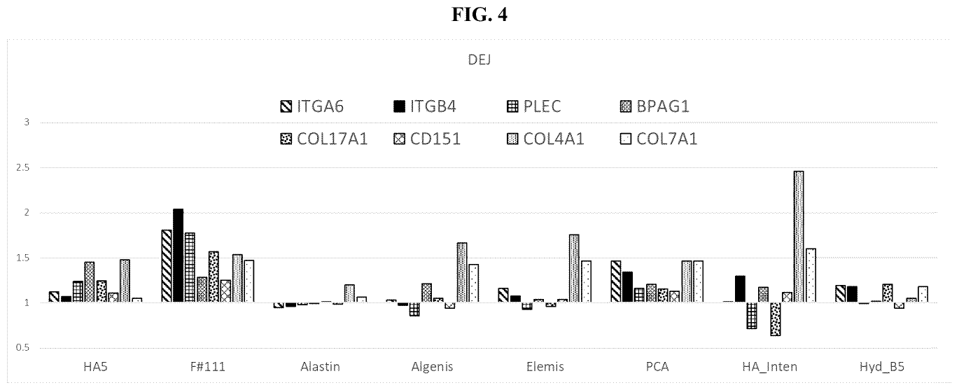

In vitro studies employing a topical composition as described in Example 2 were conducted to determine the efficacy of the topical compositions of the present invention. In vitro studies were conducted using 3-D cell culture models as generally described in Naughton et al., “Targeting Multiple Hallmarks of Skin Aging: Preclinical and Clinical Efficacy of a Novel Growth Factor-Based Skin Care Serum,” Dermatology and Therapy, 2023 January; 13 (1): 169-86., incorporated herein by reference. A composition as described in Example 2 and in accordance with the present invention was tested against six commercially available anti-aging products. The results of the HA and proteoglycan gene test panel is shown in FIG. 1. The results of the barrier function gene test panels are shown in FIG. 2A (cell-cell adhesion gene test panel), FIG. 2B (cornified envelope gene test panel) and FIG. 2C (lipid synthesis gene test panel). The results of the antioxidant gene test panel is shown in FIG. 3. The results of the hemidesmosome (DEJ) gene test panel is shown in FIG. 4.

The composition of Example 2 is referred to as F #111 in FIGS. 1-4.

The SKINMEDICA® HA⁵® product is reported to have the following ingredients: water, dimethicone, HDI/trimethylol hexyllactone crosspolymer, glycerin, butylene glycol, polysilicone-11, bis-PEG-8 dimethicone, sodium acrylate/sodium acryloyldimethyl taurate copolymer, sodium hyaluronate crosspolymer, Vitis vinifera (grape) flower cell extract, Vibrio alginolyticus ferment filtrate, Porphyridium creuentum extract, whey protein, plankton, trehalose, urea, serine, algin, caprylyl glycol, pullulan, disodium phosphate, potassium phosphate, pentylene glycol, polymethylsilsesquioxane, glyceryl polyacrylate, sodium citrate, sea water, sucrose palmitate, tocopheryl acetate, hydroxyacetophenone, polysorbate 60, propanediol, potassium sorbate, citric acid, isohexadecane, polysorbate 80, silica, decyl glucoside, tromethamine, ethylhexylglycerin, phenoxyethanol, disodium EDTA.

The ALASTIN® HA (Hyaluronic acid) IMMERSE SERUM™ is reported to have the following ingredients: water, glycerin, polyacrylate-13, sodium hyaluronate, sodium hyaluronate crosspolymer, octapeptide-45, hexapeptide-11, Tremella fuciformis sporocarp (silver car mushroom) extract, lactoferrin, tetradecyl aminobutyroylvalylaminobutyric urea trifluoroacetate, xylitol, propanediol, anhydroxylitol, phospholipids, xylitylglucoside, phosphatidylserine, glucose, hydroxymethoxyphenyl decanone, tocopherol, ascorbyl palmitate, dimethicone, caprylyl methicone, caprylic/capric triglyceride, polyisobutene, caprylyl glycol, disodium EDTA, betaine, polysorbate 20, ethylhexylglycerin, caprylhydroxamic acid, sorbitan isostearate, Helianthus annuus (sunflower) seed oil, lecithin, pentylene glycol, magnesium chloride, phenoxyethanol, potassium sorbate and sodium hydroxide.

The ALGENIST®'s GENIUS LIQUID COLLAGEN® product is reported to have the following ingredients: collagen, water, propanediol, isononyl isononanoate, butylene glycol, glycerin, betaine, pentylene glycol, dextrin palmitate, collagen amino acids, Parachlorella beijerinckii exopolysaccharides, Chlorella protothecoides oil, Helichrysum stoechas flower extract, Cylindrotheca fusiformis extract, niacinamide, tocopherol, palmitoyl tripeptide-1, palmitoyl tetrapeptide-7, adenosine, Helianthus annuus (sunflower) seed oil, Leuconostoc/radish root ferment filtrate, caprylic/capric triglyceride, palmitic acid, disodium EDTA, carbomer, sodium lactate, amodimethicone, homosalate, polysorbate 20, butyl methoxydibenzoylmethane, 1,2-hexanediol, caprylyl glycol, octocrylene, sodium hydroxide, phenoxyethanol, fragrance, limonene, and beta-carotene.

The ELEMIS® PRO-COLLAGEN MARINE CREAM product is reported to have the following ingredients: water, glycerin, caprylic/capric triglyceride, glyceryl stearate, isononyl isononanoate, dicaprylyl carbonate, dimethicone, phenoxyethanol, polyacrylate-13, cetyl alcohol, stearic acid, tocopheryl acetate, coco-caprylate/caprate, xanthan gum, polyisobutene, fragrance, tocopherol, Butyrospermum parkii (shea butter), chlorphenesin, Triticum vulgare (wheat) germ oil, Chlorella vulgaris extract, glyceryl polyacrylate, Daucus carota sativa (carrot) root extract, glyceryl acrylate/acrylic acid copolymer, disodium EDTA, Padina pavonica thallus extract, sodium dehydroacetate, polysorbate 20, sorbitan isostearate, Ginkgo biloba leaf extract, Porphyridium cruentum extract, Mimosa tenuiflora bark extract, Rosa damascena flower extract, collagen amino acids, linalool, citronellol, Leuconostoc/radish root ferment filtrate, potassium sorbate, sodium benzoate, limonene, geraniol and citric acid.

The PCA SKIN® COLLAGEN HYDRATOR CREAM is reported to have the following ingredients: water, glyceryl stearate, glycerin, C12-15 alkyl benzoate, cetearyl alcohol, isopropyl palmitate, cyclopentasiloxane, Butyrospermum parkii (shea butter), dimethicone, cetyl alcohol, polysorbate 60, tetrahexyldecyl ascorbate, tocopherol, hydrolyzed wheat protein, Chamomilla recutita (matricaria) flower/leaf extract, aloe barbadensis leaf extract, Cucumis sativus (cucumber) fruit extract, Prunus amygdalus dulcis (sweet almond) oil, Helianthus annuus (sunflower) seed oil, Olea europaea (olive) fruit oil, sodium hyaluronate, sodium PCA, allantoin, potassium cetyl phosphate, glycol distearate, xanthan gum, phenoxyethanol, ethylhexylglycerin, sodium hydroxide, carbomer and tetrasodium EDTA.

The SKINCEUTICALS® Hyaluronic Acid Intensifier (H.A.) product is reported to have the following ingredients: water, cyclohexsiloxane, glycerin, alcohol denatured, hydroxypropyl tetrahydropyrantriol, propylene glycol, dipotassium glycyrrhizate, polysilicone-11, polymethylsilsesquioxane, sodium hyaluronate, dimethicone, tocopherol, phenoxyethanol, capryloyl salicylic acid, octyldodecanil, bis-PEG/PPG-16/16/PEG/PPG-16/16 dimethicone, PEG-20 methyl glucose sesquistearate, ammonium polyacryloyldimethyl taurate, caprylyl/capric triglyceride, sodium hydroxide, adenosine, Citrus nobilis peel oil/mandarin orange peel oil, limonene, t-butyl alcohol, cellulose acetate butyrate, polyphosphorylcholine glycol acrylate, polyvinyl alcohol, sodium chloride, butylene glycol, and pentaerythrityl tetra-di-t-butyl hydroxyhydrocinnamate.

The SKINCEUTICALS® Hydrating B5 Gel product is reported to have the following ingredients: water, phenoxyethanol, calcium pantothenate and sodium hyaluronate.

The results of the in vitro testing shown in FIGS. 1-4 are summarized as follows:

| | | BARRIER | | | |
|---|---|---|---|---|---|
| | HA & PG | FUNCTION | AOX | DEJ | OVERALL |
| Example 2 (F#111) | **** | ***** | **** | ***** | 4.5 |
| SKINMEDICA ® | *** | ** | **** | ** | 3 |
| HA⁵ ® | | | | | |
| ALASTIN ® HA | * | * | * | * | 1 |
| (Hyaluronic acid) | | | | | |
| IMMERSE | | | | | |
| SERUM ™ | | | | | |
| ALGENIST ®'s | ** | *** | ** | * | 2 |
| GENIUS LIQUID | | | | | |
| COLLAGEN ® | | | | | |
| ELEMIS ® PRO- | *** | ** | ** | * | 2 |
| COLLAGEN | | | | | |
| MARINE CREAM | | | | | |
| PCA SKIN ® | *** | ** | ** | ** | 2.25 |
| COLLAGEN | | | | | |
| HYDRATOR | | | | | |
| CREAM | | | | | |
| SKINCEUTICALS ® | **** | *** | ** | ** | 2.75 |
| Hyaluronic Acid | | | | | |
| Intensifier (H.A.) | | | | | |
| SKINCEUTICALS ® | * | ** | ** | * | 1.5 |
| Hydrating B5 Gel | | | | | |

The data in FIGS. 1-4 and summarized in the above table shows the compositions in accordance with the present invention exhibit improved hydration, improved cell-cell adhesion, improved barrier function, improved antioxidant properties and enhanced DEJ properties. The grading 0-5* is based on the beneficial gene expression levels of the multiple target biomarkers for each category.

The invention described herein may be practiced in the absence of any element or limitation which is not specifically disclosed herein. Thus, for example, in each instance herein, any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A topical composition comprising: (i) one or more collagens; (ii) one or more barrier support agents; (iii) one or more bio-hydration support agents; (iv) one or more dermal-epidermal junction agents; (v) one or more antioxidants; and (vi) hyaluronic acid, hyaluronic acid derivatives or combinations of hyaluronic acid and/or hyaluronic acid derivatives.

2. The topical composition of claim 1 that is a solution, suspension, dispersion, emulsion, gel, cream, lotion, ointment or serum.

3. The topical composition of claim 1 further comprising one or more cosmetic excipients selected from solvents, skin conditioning agents, preservatives, viscosity enhancing agents, pH adjusting agents, buffering agents, chelating agents, surfactants, solubilizing agents or emulsifying agents and combinations of the foregoing.

4. A topical composition comprising: (i) about 0.0001 wt % to about 2.5 wt % of one or more collagens; (ii) about 0.0001 wt % to about 5 wt % of one or more barrier support agents; (iii) about 0.0001 wt % to about 5 wt % of one or more bio-hydration support agents; (iv) about 0.0001 wt % to about 5 wt % of one or more dermal-epidermal junction agents; (v) about 0.001 wt % to about 2.5 wt % of one or more antioxidants; and (vi) about 0.001 wt % to about 2.5 wt % of hyaluronic acid, hyaluronic acid derivatives or combinations of hyaluronic acid and/or hyaluronic acid derivatives.

5. The topical composition of claim 4 further comprising one or more excipients selected from the group consisting of solvents, skin conditioning agents, preservatives, viscosity enhancing agents, pH adjusting agents, buffering agents, chelating agents, surfactants, solubilizing agents or emulsifying agents and combinations of the foregoing.

6. The topical composition of claim 4 further comprising at least about 60 wt % of water.

7. The topical composition of claim 4 wherein the one or more collagens comprise collagen 21.

8. The topical composition of claim 4 wherein the one or more barrier support agents comprises linoleic acid, linolenic acid or a combination thereof.

9. The topical composition of claim 4 wherein the one or more bio-hydration support agents comprises a botanical 5α reductase inhibitor.

10. The topical composition of claim 4 wherein the one or more dermal-epidermal junction agents comprise Passiflora edulis seed oil.

11. The topical composition of claim 4 wherein the one or more antioxidants comprise a botanical extract selected from Nelumbo nucifera germ extract, Vitis vinifera (grape) flower extract and combinations thereof.

12. The topical composition of claim 4 wherein the hyaluronic acid, hyaluronic acid derivatives or combinations of hyaluronic acid and/or hyaluronic acid derivatives comprise sodium hyaluronate, hydrolyzed hyaluronic acid and sodium hyaluronate crosspolymer.

13. The topical composition of claim 4 further comprising one or more anti-inflammatory agents and at least 85 wt % of water.

14. The topical composition of claim 12 used concurrently with a microdermabrasion procedure.

15. A topical composition comprising:
0.001 wt % to 1.5 wt % of one or more collagens;
0.0005 wt % to 2 wt % of one or more barrier support agents comprising linoleic acid, linolenic acid, or combinations thereof;
0.0005 wt % to 2 wt % of one or more bio-hydration support agents comprising a botanical 5α reductase inhibitor comprising butyl avocadate, saw palmetto extract, black pepper extract or combinations thereof;
0.0005 wt % to 2 wt % of a dermal-epidermal junction support agent comprising Passiflora edulis seed oil;
0.005 wt % to 1.5 wt % of one or more antioxidants comprising Nelumbo nucifera germ extract, Vitis vinifera (grape) flower extract or combinations thereof;
0.005 wt % to 1.5 wt % of hyaluronic acid, hyaluronic acid derivatives, or combinations thereof comprising sodium hyaluronate, hydrolyzed hyaluronic acid, sodium hyaluronate crosspolymer or combinations thereof.

16. The topical composition of claim 15 wherein the one or more collagens comprise collagen 21.

17. The topical composition of claim 15 further comprising one or more anti-inflammatory agents and at least 70 wt % of water.

18. The topical composition of claim 17 used concurrently with a microdermabrasion procedure.

19. The topical composition of claim 15 further comprising one or more of the following: water, glycerin, caprylic/capric triglyceride, Shorea stenoptera seed butter, Butyrospermum parkii (shea) butter, C₉-C₁₂ alkanes, propanediol, sodium acrylates copolymer, glyceryl glucoside, 1,2-hexanediol, lecithin, trehalose, urea, oleic acid, palmitic acid, coco-caprylate/caprate, pentylene glycol, caprylhydroxamic acid, serine, stearic acid, polyglutamic acid, algin, capryl glycol, glyceryl polyacrylate, pullulan, one or more polysorbates, potassium phosphate, tocopherol, sodium benzoate, aloe barbadensis leaf extract, one or more vitamin B and/or provitamin B (e.g. panthenol), allantoin, phenoxyethanol, chlorphenesin, caprylyl glycol, PPG-2 isoceteth 20 acetate or combinations thereof.
